(19) United States
(12) Patent Application Publication
(10) Pub. No.: US 2017/0066793 A1
(43) Pub. Date: Mar. 9, 2017

(54) METHOD FOR THE ISOMERIZATION OF GLUCOSE TO FRUCTOSE

(71) Applicants: Jean-Philippe Tessonnier, Ames, IA (US); Chi Liu, Ames, IA (US); Jack M. Carraher, Ames, IA (US)

(72) Inventors: Jean-Philippe Tessonnier, Ames, IA (US); Chi Liu, Ames, IA (US); Jack M. Carraher, Ames, IA (US)

(21) Appl. No.: 15/342,934

(22) Filed: Nov. 3, 2016

Related U.S. Application Data
(63) Continuation of application No. PCT/US2015/028059, filed on Apr. 28, 2015.
(60) Provisional application No. 61/989,181, filed on May 6, 2014.

(51) Int. Cl. C07H 3/02; C07H 1/06

(52) U.S. Cl. CPC C07H 3/02; C07H 1/06

(57) ABSTRACT

In various embodiments, the present invention provides methods to isomerize glucose to fructose using abuse. In one embodiment, the method includes catalyzing isomerization of glucose to fructose including combining an effective catalytic amount of a base with glucose in an aqueous medium so that the glucose is isomerized to yield a mixture comprising fructose and glucose.

### CROSS-REFERENCE TO RELATED APPLICATIONS

This application is a 35 U.S.C. §111(a) continuation and claims the benefit of priority of PCT/US2015/028059, filed Apr. 28, 2015, and published in English on Nov. 12, 2015 as WO 2015/171368, which claims the benefit of priority to U.S. Provisional Patent Application Ser. No. 61/989,181, filed May 6, 2014, the benefit of priority of each of which is claimed hereby, and each of which are incorporated herein in their entirety by reference.

### GOVERNMENT GRANT SUPPORT

This invention was made with the support of the National Science Foundation under grant number EEC-0813570. The Government has certain rights in the invention.

### BACKGROUND OF THE INVENTION

The isomerization of sugars is a key reaction used in various industrial processes. For instance, the conversion of glucose into fructose for the production of high-fructose corn syrups (HFCS) has become the largest immobilized biocatalytic process worldwide. HFCS has reached a global production exceeding 8×10⁶ tons/year (in the United States alone, per capita consumption of HFCS reached 37.8 lbs/year in 2008). See, for example, S. Bhosale et al., Microbial. Rev., 60. 280 (1996); J. Lecomte et al., Starch-Starke, 54, 75 (2002). In addition, the recent drive to use biomass as an alternative to petroleum for the production of fuels and chemical intermediates has triggered a renewed interest in carbohydrate chemistry. See, Y. Roman-Leshkov et al., Nature, 447, 982 (2007); M. Climent et al., Green Chem., 13, 520 (2011). The reaction pathway is shown in Scheme 1, below.

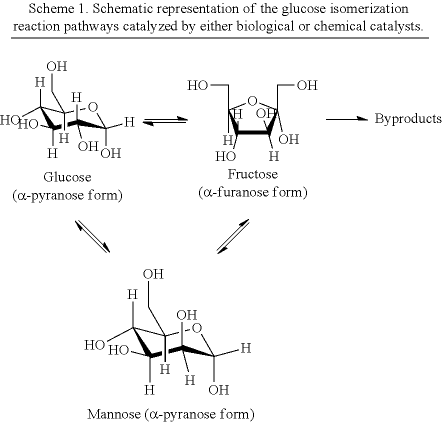

In industry, this reaction is typically catalyzed enzymatically using immobilized xylose isomerase. The highest yields of fructose reported are in the order of 42%, which is close to the thermodynamic limit for this reaction. While the enzymatic process is highly selective, it suffers major drawbacks. In particular, the reaction temperature and the pH need to be carefully controlled to assure an optimal enzymatic activity. In addition, the reaction is slow and the maximum yield of 42% typically requires about 16 to 24 hours (M. Moliner et al., PNAS. 107, 6164 (2010)).

Many research groups have recently synthesized inorganic chemical catalysts to convert glucose obtained from lignocellulosic biomass into chemicals in an integrated process. Because of its poor reactivity, glucose must first be isomerized into fructose before the fructose is converted into platform chemicals such as 5-hydroxymethylfurfural (HMF). The best catalysts reported so far are combinations of Lewis and Brønsted acids, either homogeneous and/or heterogeneous. Most catalysts transform glucose into HMF in a one-pot reaction, using a bi-phasic reactor. HMF is valuable for the production of furanics such as 2,5-furandicarboxylic acid, 2,5-bishydroxymethylfuran, 2,5-dimethylfuran, as well as organic acids such as levulinic acid. However, it would also be valuable to selectively form fructose for (i) the high fructose corn syrup industry, and (ii) to produce renewable chemicals through reaction paths that do not involve HMF as an intermediate, for example for the conversion of fructose to lactic acid. The typical reaction kinetics obtained with the xylose isomerase do not allow the integration of other chemical catalytic processes downstream.

It has been known since 1895 that Brønsted bases catalyze the isomerization of glucose to fructose. However, most groups who worked on this reaction reported very low fructose yields, in the order of 8 to 10%. These poor results are mainly due to the degradation of the sugars under strongly basic conditions. More recently, several groups have tested weak solid bases, in particular clays, metallosilicates, and other transition metal oxides. Relatively high yields of fructose were obtained, in the order of 25 to 30%. However, all these solid bases are unstable under the reaction conditions employed and dissolve during the first catalytic run. Therefore, solid bases do not present any significant advantage over homogeneous catalysts.

Historically, the Lewis acids have received more attention due to the reportedly poor selectivity and yield of the bases. See, e.g., Y. Roman-Leshkov, Angew. Chem. Int. Ed., 49, 8954 (2010). Mark Davis at the California Institute of Technology reported the first heterogeneous Lewis acid catalyst that can isomerize glucose to fructose with 31% yield within 30 minutes. Davis et al. employed a Tin-doped BETA zeolite catalyst (Sn-Beta) using a multistep procedure requiring expensive chemicals and a synthesis time of more than 40 days. See M. Moliner et al. and Y. Roman-Leshkov et al., cited supra, and E. Nikolla et al., ACS Catal., 1, 408 (2011).

Therefore, a need exists for improved methods to isomerize glucose to fructose.

### SUMMARY OF THE INVENTION

In various embodiments, the present invention provides a method of catalyzing isomerization of glucose to fructose. The method includes combining an effective catalytic amount of a base with fructose in an aqueous medium so that the glucose is isomerized to yield a mixture including fructose and glucose.

In various embodiments, the present invention provides a method of catalyzing the isomerization of glucose to fructose. The method includes combining an effective catalytic amount of an organic aliphatic amine or organic heterocyclic amine with glucose in an aqueous medium so that the glucose is isomerized to yield a mixture including fructose and glucose.

In various embodiments, the present invention provides a method of catalyzing isomerization of glucose to fructose. The method includes combining an effective catalytic amount of a base with glucose in an aqueous medium such that the initial pH of the aqueous medium is about 9 to about 12. The method also includes heating the aqueous medium to about 50-150° C., so that the glucose is isomerized to yield a mixture including fructose and glucose. The conversion of the glucose is about 50-90%. The yield of the fructose is about 20-50%. The glucose is isomerized to the fructose with about 40-80% selectivity.

In various embodiments, the present invention provides a method of catalyzing isomerization of glucose to fructose. The method includes combining an effective catalytic amount of a tri(C₁-C₁₀)alkylamine with glucose in an aqueous medium, wherein each (C₁-C₁₀)alkyl group is independently selected. The method includes heating the aqueous medium to about 50-150° C. under a substantially inert atmosphere, so that the glucose is isomerized to yield a mixture including fructose and glucose. The conversion of the glucose is about 10-100%. The yield of the fructose is about 20-50%. The glucose is isomerized to the fructose with about 40-80% selectivity.

### BRIEF DESCRIPTION OF THE FIGURES

FIG. 1 is a graph depicting the conversion, selectivity, yield, and pKa for the isomerization of glucose (10 wt-% in water) to fructose using six organic amine catalysts at a loading of 10 mol-% of the glucose, using a reaction time of 30 minutes at 100° C., in accordance with various embodiments.

FIG. 2 is a graph comparing the percent conversion of glucose, and the selectivity and yield of fructose when glucose is isomerized using triethylamine as the catalyst under the conditions of FIG. 1, in accordance with various embodiments.

FIG. 3 illustrates glucose conversion, fructose selectivity, and fructose yield versus pH, in accordance with various embodiments.

FIG. 4 illustrates conversion of glucose and yield of fructose under various conditions, in accordance with various embodiments.

FIG. 5 illustrates a UV spectrum of a solution including 10 wt % glucose with various bases after a 15 minute reaction, in accordance with various embodiments.

FIG. 6 illustrates photographs and UV spectra of a byproduct after generation (left), after addition of acid (middle), and after addition of base (right), in accordance with various embodiments.

FIG. 7 illustrates glucose conversion and pH versus time, in accordance with various embodiments.

FIG. 8A illustrates a mechanism of glucose isomerization to fructose, in accordance with various embodiments.

FIG. 8B illustrates degradation pathways during base-catalyzed of glucose isomerization to fructose, in accordance with various embodiments.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible Manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should he interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” “the” are used to include one or more than one unless the context clearly dictates otherwise, The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

A wide variety of aliphatic amines, heterocyclic amines and heteroaryl amines can be employed in the present method, and can be selected to be non-toxic, sufficiently basic and water soluble. Aliphatic and heterocyclic amines can be as catalysts, and mixtures of two or more of the organic amines found to be effective singly, can be employed.

Broadly defined, useful organic amines include compounds of the structure N(Rₐ)(R_{b})(R_{c}), wherein each of Rₐ, R_{b} and R_{c} is independently H, (C₁-C₆)alkyl, (C₃-C₆)cycloalkyl, (C₆-C₁₀)aryl, heteroaryl, or heterocyclyl; Rₐ and R_{b} together with the nitrogen to which they are attached forma heterocyclic ring or a heteroaromatic ring; with the proviso that at least one of Rₐ, R_{b} or R_{c} is not hydrogen.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The terms “heteroaryl” and “heterocyclyl” refer to a monovalent heteroaromatic ring or heterocyclic ring. A heterocyclic ring encompasses a monocyclic, bicyclic, or tricyclic ring system containing a total of 3-20 atoms, including at least one N(H) moiety and one or more (e.g., 1, 2, 3, 4, 5, or 6) carbon atoms, and optionally, one or more (e.g., 1, 2, 3, or 4) heteroatoms selected from oxygen, sulfur, and N(X) wherein X is absent or is H, O, (C₁-C₄)alkyl, phenyl or benzyl, wherein one or more ring carbons of the heterocyclic ring can optionally be substituted with oxo (═O).

A heteroaromatic ring encompasses a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a monovalent ortho-fused bicyclic heterocycle. of about eight to ten ring atoms derived therefrom, particularly a benz-derivative or one derived by fusing a propylene, trimethylene, or tetramethylene substituent thereto.

Any (C₁-C₆)alkyl is optionally substituted with one or more (e.g., 1, 2, 3, or 4) halo, hydroxy, (C₁-C₆)alkoxy, (C₃-C₆)cycloalkyloxy, (C₁-C₆)alkanoyl, (C₁-C₆)alkanoyloxy, trifluoromethyl, azido, cyano, oxo (═O), (C₁-C₆)alkyl, (C₃-C₆)cycloalkyl, (C₃-C₆)cycloalkyl(C₁-C₆)alkyl, (C₁-C₆)alkyl-S—(C₁-C₆)alkyl-, aryl, heteroaryl, aryl(C₁-C₆)alkyl, or heteroaryl(C₁-C₆)alkyl, NRₐⱼRₐₖ; wherein each Rₐⱼ and Rₐₖ is independently hydrogen, (C₁-C₆)alkyl, (C₃-C₆)cycloalkyl, phenyl, benzyl, or phenethyl.

Any aryl, heteroaryl, aromatic ring or heterocyclic ring may optionally be substituted with one or more substituents selected from the group consisting of halo, hydroxy, (C₁-C₆)alkyl, (C₃-C₆)cycloalkyl, (C₁-C₆)alkoxy, (C₃-C₆)cycloalkyloxy, (C₁-C₆)alkanoyl, (C₁-C₆)alkanoyloxy, trifluoromethyl, trifluoromethoxy, nitro, cyano, and amino.

In various embodiments, the catalyst can be a (C₁-C₆)₃N or 5- or 6-membered heterocyclic rings including 1 or 2 N(X) wherein at least one X is H. In various embodiments, the amine can be a tertiary amine such as a tris(aliphatic) amine. Suitable amines can include triethylamine (TEA), pyrrolidine, piperidine, piperazine and morpholine.

As used herein the term “aqueous medium” refers to water that may contain up to about 50 vol % of co-solvents (e.g., DMSO or an alcohol), surfactants, pH-adjusting agents, stabilizers and the like for the catalyst(s), the glucose or the fructose.

### Method of Isomerizing Glucose to Fructose.

In various embodiments, the present invention provides a method of catalyzing isomerization of glucose to fructose. The method can include combining an effective catalytic amount of a base with glucose in an aqueous medium so that the glucose is isomerized to yield a mixture including fructose and glucose.

The base can be any suitable base, so long as the base does not catalyze the formation of undesired byproducts, and so long as the pH is sufficiently high to deprotonate the glucose but not so high that pH-mediated degradation occurs rather than fructose production. The base can be an amine, such as any of a wide variety of aliphatic, heterocyclic and heteroaromatic amines. The base can be an organic aliphatic amine or organic heterocyclic amine. The aliphatic amine can be a tri(C₁-C₁₀)alkylamine, wherein each (C₁-C₁₀)alkyl group is independently selected, such as triethylamine. Tertiary aliphatic amines, such as tris(lower alkyl) amines, e.g., tris(C₁-C₄)alkyl amines, can be used. Primary and secondary amines can cause the undesired Maillard reaction. In some embodiments, the base can be an inorganic base, such as NaOH, KOH, LiOH, or a combination thereof. In some embodiments, the base can be substantially free of calcium or magnesium salts or bases, such as Ca(OH)₂ and Mg(OH), due to interactions between calcium or magnesium ions and ring-opened intermediates. In some embodiments, the base can be a combination of an inorganic base and an organic base.

The amount or concentration of the base in the aqueous medium is sufficient such that the pH of the aqueous medium is about 9 to about 14, about 9 to about 12, about 9.5 to about 11.5, or about 9 or less, or less than, equal to, or greater than about 9.5, 10, 10.5, 10.6, 10.7, 10.8, 10.9, 11, 11.1, 11.2, 11.3, 11.4, 11,5, 12, 12.5, 13, 13.5, or about 14 or more, wherein in various embodiments, the aqueous medium can have the pH at the time of addition of the glucose to the aqueous medium (e.g., initial pH). The mol-% of the base to the glucose can be about 2-30 mol-%, or about 7.5-12.5 mol-%, or about 2 mol-% or less, or less than, equal to, or greater than about 3 mol-%, 4, 5, 6, 7. 7.5, 8, 8.5, 9, 9.5, 10, 10.5, 11, 11.5, 12, 12.5, 13, 14, 15, 16, 17, 18, 19, 20, 21, 22, 23, 24, 25, 26, 27, 28, 29, or about 30 mol-% or more.

The reaction can proceed under mild reaction conditions. Heat is applied, e.g., externally. The method can include heating the aqueous medium to about 50-150′C., about 70-120° C., or about 50° C. or less, or less than, equal to, or greater than about 55, 60, 65, 70, 75, 80, 85, 90, 95, 100, 105, 110, 115, 120, 125, 130, 135, 140, 145, or about 150° C. or more. Reaction times can be about 1 minute to about 2.0 hours, such as from about 2 minutes to about 30 minutes, about 2 minutes to about 10 minutes, or about 5 minutes to about 9 minutes, or about 7 minutes, or 1 minute or less, or less than, equal to, or greater than about 2 minutes, 3, 4, 5, 6, 7, 8, 9, 10, 12, 14, 16, 18, 20, 25, 30, 35, 40, 45, 50, 55, 1 hour, 1.1, 1.2, 1.3, 1.4, 1.5, 1.6, 1.7, 1.8, 1.9, or about 2 h or more. The method can include agitating the aqueous solution during the isomerization, such as with stirring. High pressures are not required.

The isomerization can be carried out under any suitable atmospheric conditions. The isomerization can be carried out under an inert atmosphere (e.g., substantially inert), such as under argon, nitrogen, or a combination thereof, to exclude CO₂. The isomerization can be carried out under ambient conditions. In various embodiments, the produced fructose can be removed from the system immediately after formation or shortly after formation to help avoid degradation.

The initial concentration of the glucose can be any suitable concentration, such as about 0.001 wt % to about 65 wt %, or about 5 wt % to about 15 wt %, or about 0,001 wt % or less, or less than, equal to, or greater than about 0.01 wt %, 0.1, 1, 2, 3, 4, 5, 6, 7, 8, 9, 10, 11, 12, 13, 14, 15, 16, 18, 20, 25, 30, 40, 45, 50, 55, 60, 60, or about 65 wt % or more.

The method can isomerize glucose to fructose with any suitable selectivity, such as about 30-90% selectivity, 40-80% selectivity, about 50-70% selectivity, or about 30% or less, or less than, equal to, or greater than about 32, 34, 36, 38, 40, 42, 44, 46, 48, 50, 52, 54, 56, 58, 60, 62, 64, 66, 68, 70, 72, 74, 76, 78, or about 80% or more. The method can have any suitable percent conversion of the glucose, such as about 10-100%, about 30-90%, about 25-85%, about 55-80%, or about 10% or less, or less than, equal to, or greater than about 15%, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 85, 90, or about 95% or more. The selectivity and percent conversion to fructose can be measured by methods known to the art. See, e.g., S. Yu et al., Catal. Commun., 29, 63 (2012) (HPLC); UPLC, see below, and M. Moliner et al., PNAS. 107, 6164 (2010).

The method can provide any suitable yield of fructose from glucose, such as about 10% to about 60%, about 20-50%, about 30-40%, or about 10% or less, or less than, equal to, or greater than about 10%, 15, 20, 25, 30, 35, 40, 45, 50, 55, or about 60% or more.

The glucose can be obtained from carbohydrate components of biomass, such as starch or celluose.

The fructose can be recovered and converted to other useful chemicals, such as at least one of 5-hydroxymethylfurfural (HMF), 2,5-furandicarboxylic acid (FDCA), and levulinic acid.

The method can include treating the fructose with activated carbon to remove colored impurities. In some embodiments, removal of color-generating materials in the fructose can be avoided or can be supplemented with an adjustment of the pH of the fructose-containing product to substantially eliminate yellowing. By making the pH more acidic, deprotonated species that cause a yellow color can be caused to stop generating color. The pH can be adjusted as suitable to eliminate the color, such as to about 4-10, about 5-9, or to about 4 or less, or to less than, equal to, or greater than about 4,5, 5, 5.5, 6, 6.5, 7, 7.5, 8, 8.5, 9, 9,5, or about 10, to substantially eliminate yellowing.

### EXAMPLES

### Example 1

For all the Samples listed on Tables 1, 2 and 3, a 10 wt-% glucose solution in water was inserted into 5 ml glass vials, with disposable plastic Teflon lined screw caps (Chunglass Life Sciences NJ, USA). The solution was measured and transferred using an electronic pipet (Repeater Stream, Eppendorf NY, USA).

Homogeneous organic or inorganic bases triethylamine (≧99% Sigma-Aldrich MO, USA) (TEA), pyridine (99.8% Sigma-Aldrich MO, USA), pyrrolidine (99.5% Sigma-Aldrich MO, USA), piperazine (99% Sigma-Aldrich MO, USA), sodium Hydroxide (99.2% Fisher Scientific MA, USA) morpholine (≧99% Sigma-Aldrich MO, USA), or piperidine (99% Sigma-Aldrich MO, USA), were loaded at 2, 5, 10, 20, 30 mol % in respect to glucose molar concentration. The reaction vials were then sealed and heated using a stirring/heating plate (RCT Basic, IKA Lab Technologies NC, USA) equipped with a 21-well heating block (Chunglass Life Sciences NJ, USA), A stirring rate of 500 rpm was used to promote mass transfer during reaction. A small amount of silicon oil was deposited in each well to minimize the heat transfer limitation from the heating block to the reaction vials.

The vials were quenched in an ice bath after the desired reaction time was reached. A 0.5 ml aliquot was taken from each vial for analysis. A dilution of 1:20 was then made by combining the aliquot and 50/50 water/acetonitrile diluent. The conversion of glucose and generation of fructose was measured using an ultra performance liquid chromatography-evaporative light scattering detector system (UPLC-ELSD) (Waters Mass., USA), equipped with a Waters UPLC column (Acquity UPLC BEH Amide 1.7 μm, 2.1×100 mm). Data was collected and analyzed using Waters Empower 3 Software.

TABLE 1
Reaction in 10 wt-% glucose solution

| | | | | | Mol % | | |
|---|---|---|---|---|---|---|---|
| | | | | | Loading | Glu- | |
| | | | | | of cata- | cose | Fruc- |
| Sam- | | | | Temper- | lyst to | Conver- | tose |
| ple | Catalyst | Time | | ature | glucose | sion | Yield |
| 1-1a | TEA | 1 | h | 100° C. | 2 | 26.43% | 20.58% |
| 1-1b | TEA | 1 | h | 100° C. | 5 | 44.63% | 26.55% |
| 1-1c | TEA | 1 | h | 100° C. | 10 | 56.98% | 27.87% |
| 1-1d | TEA | 1 | h | 100° C. | 20 | 72.09% | 25.44% |
| 1-1e | TEA | 1 | h | 100° C. | 30 | 79.79% | 21.42% |
| 1-2a | NaOH | 1 | h | 100° C. | 5 | 49.62% | 27.73% |
| 1-2b | NaOH | 1 | h | 100° C. | 10 | 62.78% | 27.14% |
| 1-2c | NaOH | 1 | h | 100° C. | 20 | 73.75% | 24.34% |
| 1-2d | NaOH | 1 | h | 100° C. | 30 | 81.50% | 20.38% |
| 1-3a | TEA | 30 | min | 100° C. | 2 | 31.07% | 23.04% |
| 1-3b | TEA | 30 | min | 100° C. | 5 | 44.85% | 27.49% |
| 1-3c | TEA | 30 | min | 100° C. | 10 | 56.63% | 30.73% |
| 1-3d | TEA | 30 | min | 100° C. | 20 | 68.73% | 26.61% |
| 1-3e | TEA | 30 | min | 100° C. | 30 | 75.40% | 24.52% |
| 1-4a | NaOH | 30 | min | 100° C. | 2 | 36.05% | 24.93% |
| 1-4b | NaOH | 30 | min | 100° C. | 5 | 50.08% | 29.20% |
| 1-4c | NaOH | 30 | min | 100° C. | 10 | 59.85% | 30.28% |
| 1-4d | NaOH | 30 | min | 100° C. | 20 | 72.54% | 25.89% |
| 1-4e | NaOH | 30 | min | 100° C. | 30 | 80.59% | 21.33% |
| 1-5a | Piperidine | 30 | min | 100° C. | 2 | 34.56% | 22.10% |
| 1-5b | Piperidine | 30 | min | 100° C. | 5 | 45.17% | 26.97% |
| 1-5c | Piperidine | 30 | min | 100° C. | 10 | 56.47% | 28.87% |
| 1-5d | Piperidine | 30 | min | 100° C. | 20 | 67.16% | 27.16% |
| 1-5e | Piperidine | 30 | min | 100° C. | 30 | 73.88% | 23.64% |
| 1-6a | Piperazine | 30 | min | 100° C. | 2 | 24.68% | 20.42% |
| 1-6b | Piperazine | 30 | min | 100° C. | 5 | 35.05% | 25.52% |
| 1-6c | Piperazine | 30 | min | 100° C. | 10 | 45.35% | 28.11% |
| 1-6d | Piperazine | 30 | min | 100° C. | 20 | 56.36% | 28.01% |
| 1-6e | Piperazine | 30 | min | 100° C. | 30 | 63.66% | 25.74% |
| 1-7a | Pyrollidine | 30 | min | 100° C. | 2 | 28.02% | 22.35% |
| 1-7b | Pyrollidine | 30 | min | 100° C. | 5 | 39.41% | 26.88% |
| 1-7c | Pyrollidine | 30 | min | 100° C. | 10 | 48.86% | 28.71% |
| 1-7d | Pyrollidine | 30 | min | 100° C. | 20 | 55.86% | 28.13% |
| 1-7e | Pyrollidine | 30 | min | 100° C. | 30 | 67.84% | 25.26% |
| 1-8a | TEA | 5 | min | 100° C. | 10 | 21.82% | 11.96% |
| 1-8b | TEA | 10 | min | 100° C. | 10 | 47.58% | 27.97% |
| 1-8c | TEA | 15 | min | 100° C. | 10 | 54.17% | 28.47% |
| 1-8d | TEA | 20 | min | 100° C. | 10 | 56.53% | 27.68% |
| 1-8e | TEA | 30 | min | 100° C. | 10 | 57.66% | 28.16% |
| 1-9a | TEA | 30 | min | 60° C. | 10 | 13.36% | 10.45% |
| 1-9b | TEA | 30 | min | 80° C. | 10 | 45.11% | 28.18% |
| 1-9c | TEA | 30 | min | 100° C. | 10 | 56.63% | 30.73% |
| 1-9d | TEA | 30 | min | 120° C. | 10 | 58.41% | 28.31% |

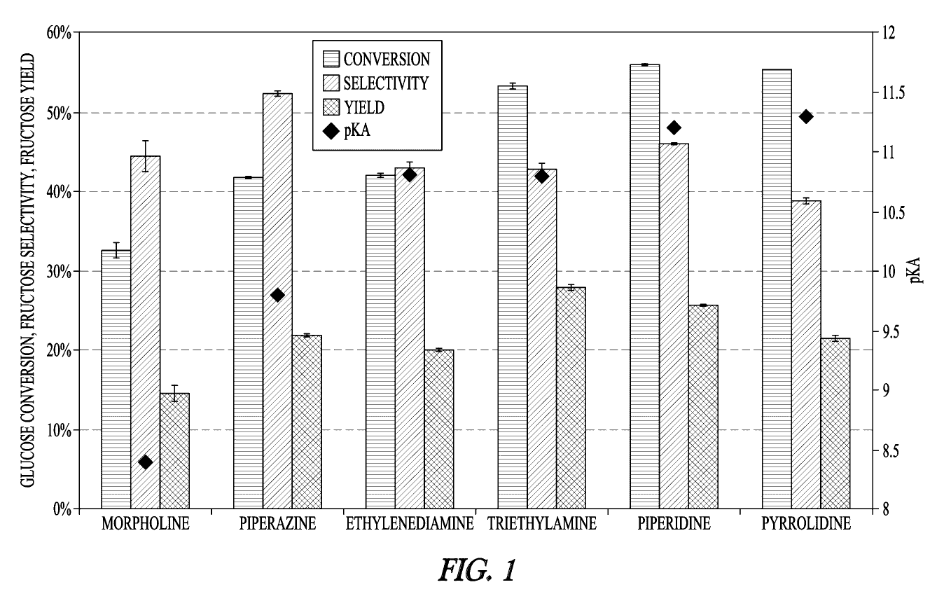

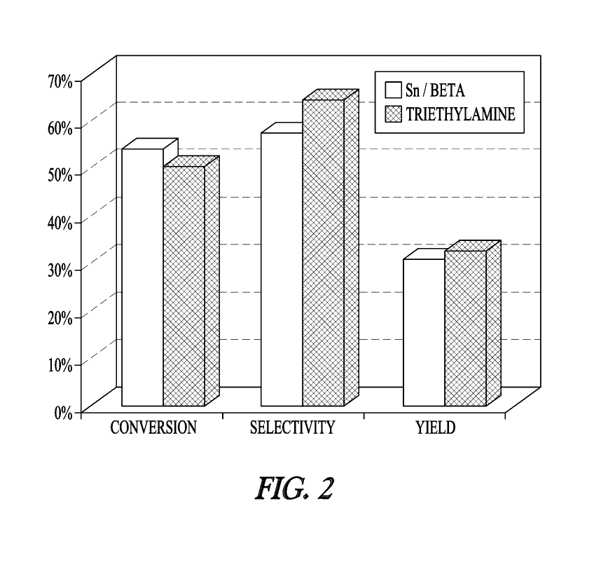

FIG. 1 is a graph summarizing the conversion, selectivity, yield, and pKa for glucose to fructose conversion for the examples of Table 1. FIG. 2 is a graph comparing the selectivity, conversion and yield attained with triethylamine used as the catalyst in the examples of Table 1 with the Sn-Beta zeolite catalysis of the Davis group. See, e.g., M. Moliner et al., PNAS, 107, 6164 (2010) wherein a 10 wt-% glucose solution was isomerized with Sn-Beta (1:50 Sn-glucose molar ratio) to yield a product that was 46 mol-% glucose, 31 mol-% fructose and 9 mol-% mannose after 30 minutes at 110° C.

Table 2 provides catalytic results using 10 mol-% loading of amine catalyst relative to glucose [10 wt-% in water] for 30 minutes at 100° C. showing glucose conversion, fructose selectivity, fructose yield and mannose yield.

TABLE 2
Catalytic results for the amine-catalyzed isomerization of
glucose to fructose. Catalytic tests performed with a 10% (wt/wt)
glucose/water solution, 10 mol. % N relative to glucose, 100°
C., 30 minutes. All results are reported within ±1%.

| Amine | Type^{a} | pKa | X_{Glu}^{b} | S_{Fru}^{c} | Y_{Fru}^{d} | Y_{Man}^{e} |
|---|---|---|---|---|---|---|
| Morpholine | Sec | 8.4 | 39 | 43 | 17 | 7 |
| Piperazine | Sec | 9.8 | 44 | 62 | 28 | 3 |
| Ethylene- | Pri | 10.8 | 42 | 60 | 25 | 3 |
| diamine | | | | | | |
| Triethylamine | Ter | 10.8 | 57 | 54 | 31 | 5 |
| Piperidine | Sec | 11.2 | 56 | 51 | 29 | 5 |
| Pyrrolidine | Sec | 11.3 | 49 | 59 | 29 | 3 |

^{a}Amine type: Pri = primary, Sec = secondary, Ter = tertiary.
^{b}Glucose conversion (%).
^{c}Fructose selectivity (%).
^{d}Fructose yield (%).
^{e}Mannose yield (%).

The data shown in Tables 1 and 2 clearly show that organic amines, and in particular triethylamine, isomerize glucose to fructose with similar selectivity (56%) and yield (28%) as Sn-Beta. See M. Moliner et al., cited supra. Differences in performance were observed for the tested amities. Glucose conversion increased with pKa, however no correlation was found between pKa and both selectivity and yield of fructose.

The Maillard reaction represents one possible pathway of byproduct formation. It is well-known in food sciences that primary and secondary amines react with reducible sugars through the Maillard reaction to form colored products. J. S. Kim et al., Food Chem., 108, 582 (2008). The kinetics involved in this non-enzymatic browning reaction are complex and vary with the structure of the amine. The Maillard reaction is significantly faster in the presence of primary amines than with secondary amines, which is consistent with the darker solution observed for ethylenediamine compared to piperidine. Tertiary amines are not expected to participate in the Maillard reaction based on the proposed reaction mechanism. See, D. D. Wirth et al., J. Pharm. Sci., 87, 31 (1998).

The Maillard reaction is particularly undesired in the present work as this stoichiometric reaction would consume both the reactant and the catalyst. Therefore, the isomerized solutions were analyzed using UV-vis spectrometry and ¹H NMR spectroscopy to elucidate the contribution from the Maillard reaction under the tested conditions. UV-vis spectra of the reacted solutions containing primary and secondary amines were red shifted relative to the spectrum of triethylamine. The magnitude of the shift was consistent with the expected kinetics: 42 nm for ethylenediamine (primary amine) and 2-14 nm for the secondary amines. The similarities between the UV-vis spectra of secondary and tertiary amines suggest that most colored byproducts observed with secondary amines are the result of thermal degradation of the carbohydrates, which is the process involved in caramelization. ¹H NMR investigations further confirmed that triethylamine does not participate in the Maillard reaction and is not consumed under reaction conditions.

The effect of temperature, reaction time, and catalyst loading were further investigated for triethylamine (Table 3). Selectivity to fructose decreased with increasing catalyst concentration and/or reaction temperature. Our results indicate that a yield of 27-32% can be obtained for a wide range of reaction conditions, making it a rather flexible process.

TABLE 3
Effect of pH and time on catalytic activity at 100° C.
All solutions were degased with argon and the reactions were
performed with an argon blanket. All results reported within ±1%.

| Sam- | Initial | Time | | | | |
|---|---|---|---|---|---|---|
| ple | pH | (min) | X_{Glu}^{a} | Y_{Fru}^{b} | S_{Fru}^{c} | Y_{Man}^{d} |
| 1-1a | 10.9 | 2 | 12.5 | 7.3 | 58.8 | 0.8 |
| 1-1b | 10.9 | 3 | 19.3 | 14.6 | 75.6 | 1.5 |
| 1-1c | 10.9 | 4 | 30.1 | 22.4 | 74.3 | 2.9 |
| 1-1d | 10.9 | 5 | 34.4 | 26.0 | 75.5 | 4.3 |
| 1-1e | 10.9 | 7 | 43.6 | 28.3 | 65.0 | 5.4 |
| 1-1f | 10.9 | 10 | 49.3 | 30.3 | 61.4 | 6.5 |
| 1-1g | 10.9 | 12 | 74.7 | 29.9 | 40.0 | 6.3 |
| 1-1h | 10.9 | 15 | 51.6 | 30.1 | 58.2 | 6.7 |
| 1-2a | 11.1 | 2 | 13.9 | 9.0 | 64.4 | 1.4 |
| 1-2b | 11.1 | 3 | 24.8 | 20.7 | 83.2 | 2.2 |
| 1-2c | 11.1 | 4 | 32.5 | 26.2 | 80.7 | 4.0 |
| 1-2d | 11.1 | 5 | 40.7 | 29.2 | 71.9 | 5.4 |
| 1-2e | 11.1 | 7 | 47.3 | 31.1 | 65.7 | 6.8 |
| 1-2f | 11.1 | 10 | 53.1 | 30.9 | 58.3 | 7.2 |
| 1-2g | 11.1 | 12 | 55.3 | 30.8 | 55.6 | 7.8 |
| 1-2h | 11.1 | 15 | 56.7 | 30.3 | 53.4 | 7.9 |
| 1-3a | 11.3 | 2 | 13.2 | 10.2 | 77.3 | 1.0 |
| 1-3b | 11.3 | 3 | 23.3 | 20.7 | 88.7 | 2.1 |
| 1-3c | 11.3 | 4 | 37.0 | 27.3 | 73.9 | 4.0 |
| 1-3d | 11.3 | 5 | 42.8 | 31.1 | 72.7 | 5.4 |
| 1-3e | 11.3 | 7 | 50.4 | 32.2 | 63.8 | 7.4 |
| 1-3f | 11.3 | 10 | 58.2 | 30.8 | 52.9 | 8.2 |
| 1-3g | 11.3 | 12 | 59.5 | 30.5 | 51.3 | 9.1 |
| 1-3h | 11.3 | 15 | 61.4 | 30.2 | 49.2 | 9.3 |
| 1-4a | 11.5 | 2 | 15.5 | 14.1 | 90.7 | 1.4 |
| 1-4b | 11.5 | 3 | 30.3 | 23.6 | 77.8 | 3.0 |
| 1-4c | 11.5 | 4 | 43.3 | 29.6 | 68.4 | 4.8 |
| 1-4d | 11.5 | 5 | 49.4 | 30.1 | 61.0 | 6.0 |
| 1-4e | 11.5 | 7 | 62.4 | 28.0 | 44.9 | 7.5 |
| 1-4f | 11.5 | 10 | 66.4 | 25.5 | 38.5 | 7.7 |
| 1-4g | 11.5 | 12 | 69.6 | 24.6 | 35.3 | 8.5 |
| 1-4h | 11.5 | 15 | 72.1 | 22.6 | 31.4 | 8.5 |

^{a}Glucose Conversion (%).
^{b}Fructose Yield (%).
^{c}Fructose Selectivity (%).
^{d}Mannose Yield (%).

Separation and purification of reaction media is often challenging, especially when homogeneous catalysts are employed. The undesired colored byproducts could be removed by simple purification using activated carbon. The solution was mixed after reaction (Table 3, Sample 1-10) with 5 wt. % of Darco® KB-G activated carbon and stirred for 1 h. The mixture was then filtered and analyzed by UV-Vis spectroscopy, UPLC, and ¹H NMR. The solution became colorless after filtration indicating that the colored byproducts adsorbed on the activated carbon.

Analysis of the purified solution by UPLC indicated that the glucose and fructose concentrations remained unchanged, meaning that the activated carbon removed the undesired colored compounds selectively. Other byproducts were not detected by ¹H NMR. Glucose, fructose, and triethylamine can be further separated by chromatography, using a similar technique as in industry to collect the fructose-rich stream (HFCS) and return the glucose- and triethylamine-rich streams to the reaction vessel. See, Rajabbeigi et al., Chem. Eng, Sci., 116, 235 (2014).

In various embodiments, the present invention provides a method to use amines to catalyze the isomerization of glucose to fructose with the same performance as state-of-the-art Lewis acid catalysts. In various embodiments, the present method employing readily available organic amine bases provides at least comparable yields and improved selectivity over Sn-Beta catalyst system when run under equivalent conditions. A yield of 32% with a selectivity to fructose of 64% were reached after 7 min at 100° C., (Table 3). Triethylamine offers several additional advantages compared to Lewis acids. First, TEA is commercially available with >99.5% purity at a low cost, $3-12/kg for bulk orders. In addition, TEA is Mdustrially produced from renewables by alkylation of ammonia with bioethanol. Finally, TEA has a relatively low toxicity and photochemically degrades within 90-240 minutes. See, e.g., BASF—The Chemical Company. Trimethyiamine Anhydrous. http://www.basf.com/group/corporate/us/en/brand/TRIMETHYLAMINE_ANHYDROUS (accessed Jul. 30, 2014); U.S. Department of Health and Human Services. National Toxixology Program—Triethylamine. http://ntp.nichs.nih.gov/go/12383 (accessed Jul. 28, 2014) and Sixty-ninth meeting of the Joint FAO/WHO Expert Committee on Food Additives (JECFA). Safety Evaluation of Certain Food Additives World Health Organization: Geneva, 2009; p 155

### Example 2

Solutions of glucose were prepared in D₂O and brought to the desired pH with NaOH and heated to 100° C. for 12 or 15 minutes. The reacted solutions were quenched in an ice bath and once cooled DMSO internal standard was added prior to collection of ¹H NMR. Tables 4 and 5 show the conditions, yield, and selectivity. Glucose conversions and fructose yields were obtained from the ¹H NMR spectra and the error associated with this method of analysis (in this specific system is anticipated to be ±2% at low glucose concentration and up to ±5% at 20-30 wt % glucose.

TABLE 4
Effect of glucose concentration on conversion and yields for 12 mol %
NaOH solutions heated to 100° C. for 12 minutes.

| Sample | wt % glu | Glu Conv | Fru Yield | Fru Selec |
|---|---|---|---|---|
| 2-1 | 0.5 | 60% | 34% | 57% |
| 2-2 | 1 | 58% | 33% | 57% |
| 2-3 | 2 | 59% | 26% | 45% |
| 2-4 | 5 | 67% | 23% | 35% |
| 2-5 | 10 | 65% | 28% | 43% |
| 2-6 | 15 | 67% | 31% | 45% |
| 2-7 | 20 | 66% | 32% | 49% |
| 2-8 | 30 | 69% | 32% | 47% |

TABLE 5
Effect of NaOH loading on glucose conversion and fructose yield for
10 wt % glucose solutions heated to 100° C. for 15 minutes.

| mol % | | | |
|---|---|---|---|
| NaOH | Glu Conv | Fru Yield | Fru Selec |
| 8 | 61% | 28% | 46% |
| 9 | 62% | 29% | 47% |
| 9.9 | 62% | 30% | 48% |
| 11.5 | 66% | 27% | 41% |
| 12 | 65% | 28% | 43% |
| 13.4 | 70% | 27% | 38% |
| 15 | 71% | 28% | 39% |
| 16.6 | 74% | 27% | 36% |
| 18.6 | 77% | 24% | 31% |

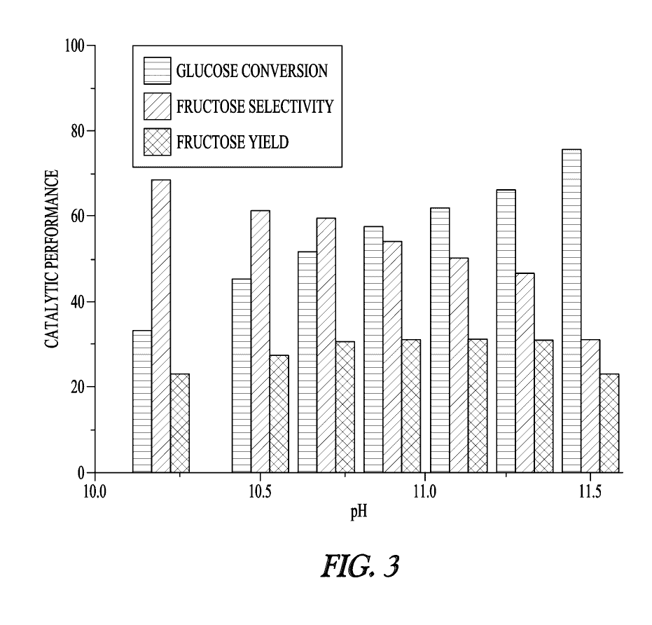

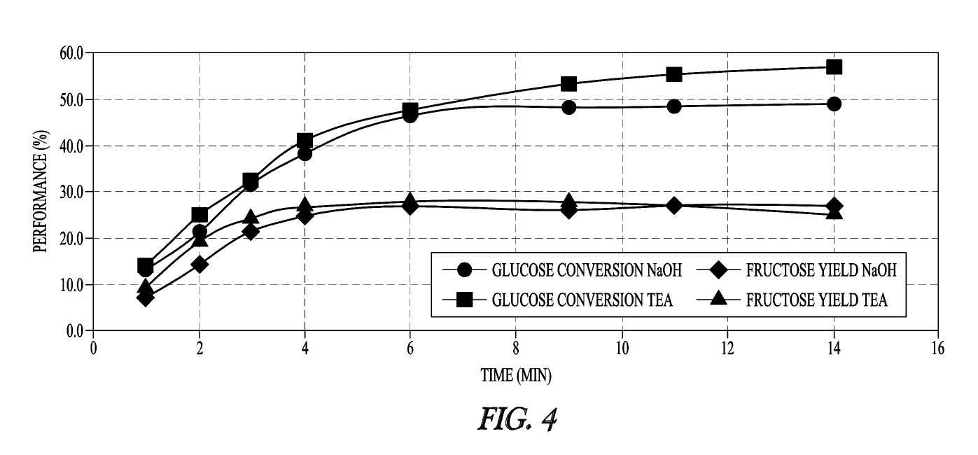

The yields shown in Tables 4 and 5 are similar to those obtained from trimethylamine, shown in FIG. 3, which illustrates catalyst performance of 10 wt % glucose with triethylamine at 100° C. for 15 minutes. FIG. 4 illustrates conversion of glucose to fructose at pH 11.1 as a function of time for NaOH and TEA catalysts, using 10 wt % glucose at 100° C., showing that kinetic traces obtained with NaOH and triethylamine each at pH 11.1 are identical. This is consistent with mechanism including unimolecular ring opening from glucose anions.

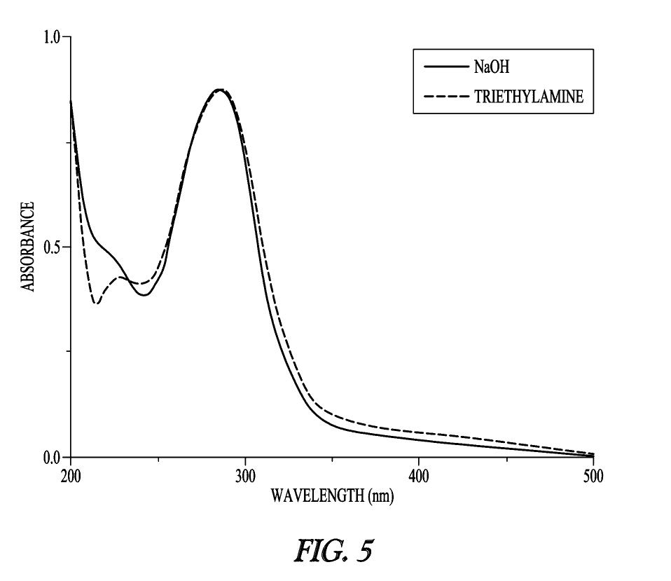

FIG. 5 illustrates a UV-vis spectrum (Shimadzn UV-2700) of colored reacted solutions of 10 wt % glucose containing NaOH or TEA at pH 11.0 reacted for 15 minutes at 100° C. The close curves in FIG. 5 indicate that the same amount of colored byproduct was formed.

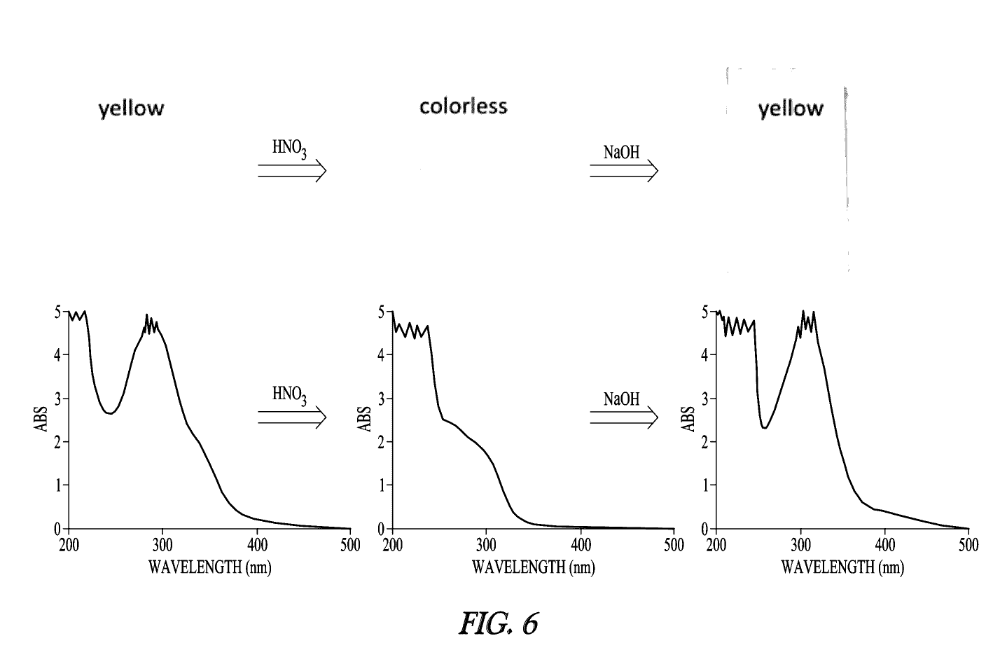

The colored byproduct can be made colorless by lowering the pH, as shown in FIG. 6. FIG. 6 illustrates pictures and UV-vis spectra (1 mm pathlength) of a 19 wt % glucose solution reacted with 12 mol % triethylamine for 15 minutes at 100° C., (left), after the addition of 1 drop of HNO₃ (middle), and after addition of a spatula tip of ground NaOH (right).

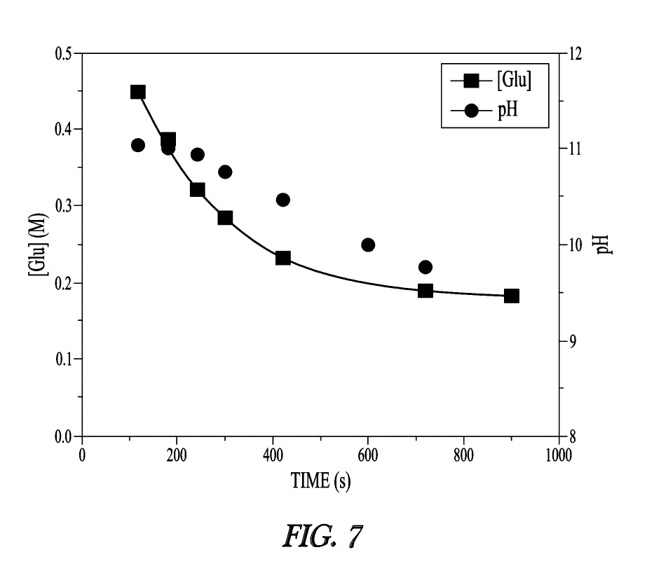

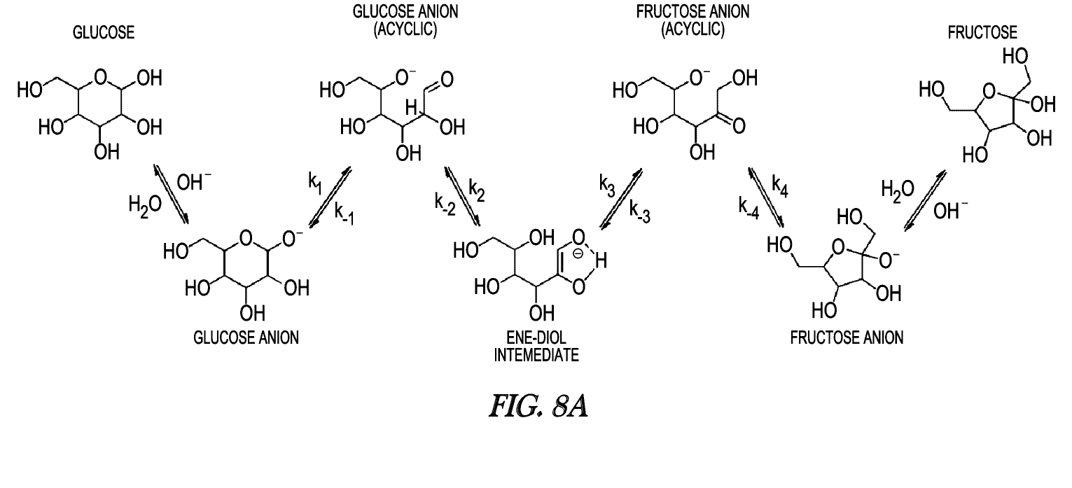

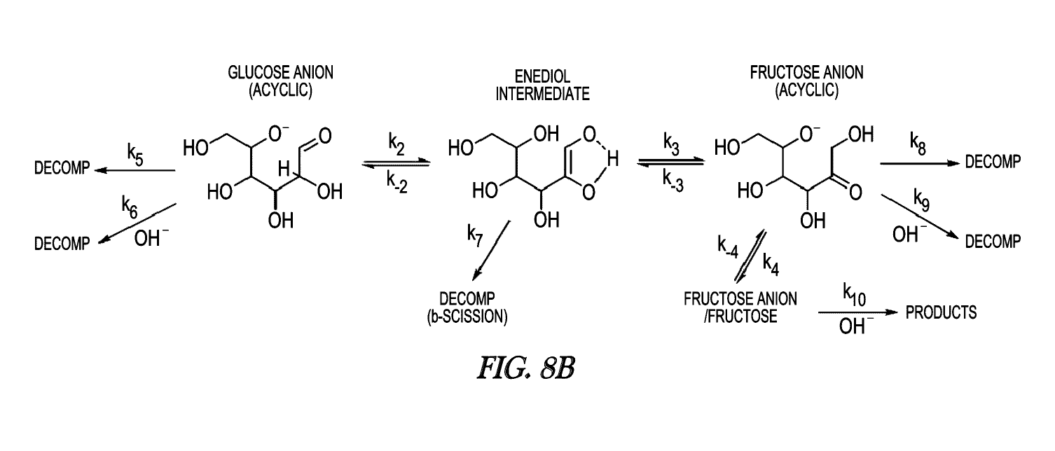

The catalytic reaction is self-quenching, such that pH decreases as organic acid derivatives of the monosaccharides are formed, as shown in FIG. 7. FIG. 7 illustrates glucose conversion and pH evolution as a function of time, using 10 wt % glucose, 100° C., and with pH₀=11.0. This is beneficial when higher yields are desired, as maintaining a high pH will degrade reactive intermediates through the pathway shown below particularly through reactive pathways k₆ and k₁₀ (FIGS. 8A-B). FIG. 8A illustrates a mechanism for isomerization of glucose to fructose using any base, provided no interactions with intermediates or side reactions occur (e.g. with secondary amines). FIG. 8B illustrates degradation pathways during base catalyzed glucose/fructose isomerization.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Additional Embodiments

The following exemplary embodiments are provided, the numbering of which is not to be construed as designating levels of importance:

Embodiment 1 provides a method comprising:

catalyzing isomerization of glucose to fructose comprising combining an effective catalytic amount of a base with glucose in an aqueous medium no that the glucose is isomerized to yield a mixture comprising fructose and glucose.

Embodiment 2 provides the method of Embodiment 1, wherein the glucose is isomerized to the fructose with about 40-80% selectivity.

Embodiment 3 provides the method of any one of Embodiments 1-2, wherein the glucose is isomerized to the fructose with about 50-70% selectivity.

Embodiment 4 provides the method of any one of Embodiments 1-3, comprising heating the aqueous medium to about 50-150° C.

Embodiment 5 provides the method of any one of Embodiments 1-4, comprising heating the aqueous medium to about 70-120° C.

Embodiment 6 provides the method of Embodiment 5, comprising heating the aqueous medium for up to about 30 minutes.

Embodiment 7 provides the method of Embodiment 6, comprising heating the aqueous medium for about 2-10 minutes.

Embodiment 8 provides the method of any one of Embodiments 1-7, wherein during the isomerizing, the aqueous medium has an initial pH of about 9 to about 14.

Embodiment 9 provides the method of any one of Embodiments 1-8, wherein during the isomerizing, the aqueous medium has an initial pH of about 9 to about 12.

Embodiment 10 provides the method of any one of Embodiments 1-9, wherein during the isomerizing, the aqueous medium has an initial pH of about 9.5 to about 11.5.

Embodiment 11 provides the method of any one of Embodiments 1-10, wherein the initial concentration of the glucose is about 5-15 wt %.

Embodiment 12 provides the method of any one of Embodiments 1-11, wherein the mol-% ratio of the base to the glucose is about 5-20 mol-%.

Embodiment 13 provides the method of any one of Embodiments 1-12, further comprising isolating and converting the fructose to at least one of 5-hydroxymethylfurfural (HMF), 2,5-furandicarboxylic acid (FDCA), and levulinic acid.

Embodiment 14 provides the method of any one of Embodiments 1-13, further comprising treating the fructose with activated carbon to remove colored impurities.

Embodiment 15 provides the method of any one of Embodiments 1-14, further comprising adjusting pH to about 4-10 to substantially eliminate yellowing.

Embodiment 16 provides the method of any one of Embodiments 1-15, further comprising adjusting pH to about 5-9 to substantially eliminate yellowing.

Embodiment 17 provides the method of any one of Embodiments 1-16, wherein the base is an organic aliphatic amine or organic heterocyclic amine.

Embodiment 18 provides the method of Embodiment 17, wherein the amine is an aliphatic amine.

Embodiment 19 provides the method of Embodiment 18, wherein the amine is a tri(C₁-C₁₀)alkylamine, wherein each (C₁-C₁₀)alkyl group is independently selected.

Embodiment 20 provides the method of any one of Embodiments 18-19, wherein the amine is triethylamine.

Embodiment 21 provides the method of any one of Embodiment 1-20, wherein the base is NaOH, KOH, LiOH, or a combination thereof.

Embodiment 22 provides the method of any one of Embodiments 1-21, wherein the isomerization is carried out under a substantially inert atmosphere.

Embodiment 23 provides the method of any one of Embodiments 1-22, wherein the yield of fructose is about 20-50%.

Embodiment 24 provides the method of any one of Embodiments 1-23, wherein the yield of fructose is about 30-40%.

Embodiment 25 provides the method of any one of Embodiments 1-24, wherein the conversion of the glucose is about 10-100%.

Embodiment 26 provides the method of any one of Embodiments 1-25, wherein the conversion of the glucose is about 30-90%.

Embodiment 27 provides the method of any one of Embodiments 1-26, wherein the conversion of the glucose is about 55-80%.

Embodiment 28 provides a method comprising:

catalyzing isomerization of glucose to fructose comprising

combining an effective catalytic amount of a base with glucose in an aqueous medium such that the initial pH of the aqueous medium is about 9 to about 12, and

heating the aqueous medium to about 50-150° C., so that the glucose is isomerized to yield a mixture comprising fructose and glucose;

wherein

the conversion of the glucose is about 50-90%,

the yield of the fructose is about 20-50%, and

the glucose is isomerized to the fructose with about 40-80% selectivity

Embodiment 29 provides a method comprising:

catalyzing isomerization of glucose to fructose comprising

combining an effective catalytic amount of a tri(C₁-C₁₀)alkylamine with glucose in an aqueous medium, wherein each (C₁-C₁₀)alkyl group is independently selected, and

heating the aqueous medium to about 50-150° C. under a substantially inert atmosphere, so that the glucose is isomerized to yield a mixture comprising fructose and glucose;

wherein

the conversion of the glucose is about 10-100%,

the yield of the fructose is about 20-50%, and

the glucose is isomerized to the fructose with about 40-80% selectivity.

Embodiment 30 provides the method of any one or any combination of Embodiments 1-29 optionally configured such that all elements or options recited are available to use or select from.

What is claimed is:

1. A method comprising:
catalyzing isomerization of glucose to fructose comprising combining an effective catalytic amount of a base with glucose in an aqueous medium no that the glucose is isomerized to yield a mixture comprising fructose and glucose.

2. The method of claim 1, wherein the glucose is isomerized to the fructose with about 40-80% selectivity.

3. The method of claim 1, comprising heating the aqueous medium to about 50-150° C.

4. The method of claim 3, comprising heating the aqueous medium for up to about 30 minutes.

5. The method of claim 4, comprising heating the aqueous medium for about 2-10 minutes.

6. The method of claim 1, wherein during the isomerizing, the aqueous medium has an initial pH of about 9 to about 14.

7. The method of claim 1, wherein the mol-% ratio of the base to the glucose is about 5-20 mol-%.

8. The method of claim 1, further comprising isolating and converting the fructose to at least one of 5-hydroxymethylfurfural (FEW), 2,5-furandicarboxylic acid (FDCA), and levulinic acid.

9. The method of claim 1, further comprising treating the fructose with activated carbon to remove colored impurities.

10. The method of claim 1, further comprising adjusting pH to about 4-10 to substantially eliminate yellowing.

11. The method of claim 1, wherein the base is an organic aliphatic amine or organic heterocyclic amine.

12. The method of claim 11, wherein the amine is an aliphatic amine.

13. The method of claim 12, wherein the amine is a tri(C₁-C₁₀)alkylamine, wherein each (C₁-C₁₀)alkyl group is independently selected.

14. The method of claim 12, wherein the amine is triethylamine.

15. The method of claim 1, wherein the base is NaOH, KOH, LiOH, or a combination thereof.

16. The method of claim 1, wherein the isomerization is carried out under a substantially inert atmosphere.

17. The method of claim 1, wherein the yield of fructose is about 20-50%.

18. The method of claim 1, wherein the conversion of the glucose is about 10-100%.

19. A method comprising:
catalyzing isomerization of glucose to fructose comprising
combining an effective catalytic amount of a base with glucose in an aqueous medium such that the initial pH of the aqueous medium is about 9 to about 12, and
heating the aqueous medium to about 50-150° C., so that the glucose is isomerized to yield a mixture comprising fructose and glucose;
wherein
the conversion of the glucose is about 50-90%,
the yield of the fructose is about 20-50%, and
the glucose is isomerized to the fructose with about 40-80% selectivity

20. A method comprising:
catalyzing isomerization of glucose to fructose comprising
combining an effective catalytic amount of a tri(C₁-C₁₀)alkylamine with glucose in an aqueous medium, wherein each (C₁-C₁₀)alkyl group is independently selected, and
heating the aqueous medium to about 50-150° C. under a substantially inert atmosphere, so that the glucose is isomerized to yield a mixture comprising fructose and glucose;
wherein
the conversion of the glucose is about 10-100%,
the yield of the fructose is about 20-50%, and
the glucose is isomerized to the fructose with about 40-80% selectivity.
